(19) United States
(12) Patent Application Publication
(10) Pub. No.: US 2007/0148681 A1
(43) Pub. Date: Jun. 28, 2007

(54) Gene synthesis kit

(75) Inventors: Anthony R. Cox, Mountain View, CA (US); Kenneth W. Mitchell, Sunnyvale, CA (US)

Correspondence Address: PERKINS COIE LLP, P.O. BOX 2168, MENLO PARK, CA, 94026, US

(73) Assignee: GENE ORACLE, INC.

(21) Appl. No.: 11/641,439

(22) Filed: Dec. 18, 2006

Related U.S. Application Data
(60) Provisional application No. 60/751,179, filed on Dec. 16, 2005.
(60) Provisional application No. 60/790,086, filed on Apr. 7, 2006.

(51) Int. Cl. C12Q 1/68; G06F 19/00

(52) U.S. Cl. 435/6; 702/20

(57) ABSTRACT

This disclosure is directed to the field of polynucleotide synthesis, and the embodiments taught herein are generally directed to a kit for use in the design of a desired polynucleotide from information obtained from a polypeptide or another polynucleotide, the generation of a custom set of oligonucleotides that complement the design, and the ordering of the custom set of oligonucleotides. The invention includes systems and methods for producing a desired polynucleotide using the kit.

### CROSS-REFERENCE

This application claims the benefit of U.S. Provisional Patent Application No. 60/751,179, filed Dec. 16, 2005, and U.S. Provisional Patent No. 60/790,086, filed Apr. 7, 2006, each of which is hereby incorporated herein in its entirety by reference.

### BACKGROUND

1. Field of the Invention

The invention is directed generally to the field of polynucleotide synthesis, and the embodiments taught herein include a kit having software for use in the design of a desired polynucleotide and preselection of a set of custom oligonucleotides used to produce the desired polynucleotide, as well as systems and methods that include the kit.

2. Description of the State of the Art

The design and production of synthetic polynucleotides can have several applications, and the availability of sequences of entire genomes has dramatically increased the number of potential protein targets, many of which will need to be overexpressed in cells other than where the DNA originate. Accordingly, gene synthesis techniques offer the potential of a fast and economically efficient approach to research and development, since the synthetic gene can be optimized for expression and constructed for easy mutational manipulation without regard to the parent genome. A problem is that the design and production of synthetic genes can not only be time-consuming for a variety of reasons, but it can also be difficult, in terms of both the knowledge required and the level of uncertainty involved.

Gene synthesis can be used, for example, in the research, development, and production of medical therapeutics. A desired polynucleotides may comprise a gene that encodes a desired protein having therapeutic applications, such that production of that protein could alleviate unnecessary pain and suffering from a disease. Or, a desired polynucleotide may work, for example, to block the biochemical pathways that result in the production of undesirable proteins that contribute to a disease, such that the act of blocking the pathway can inhibit or prevent the production of the undesirable protein. Or, the desired polynucleotide may create a protein that can act as an antigen in the production of an antibody used to treat a disease. Other applications may include industrial uses, where a particular peptide may, for example, have anticorrosion or antibacterial properties. As such, improvements in the design and production of specialized polynucleotides could be valuable in several ways to a variety of commercial operations.

One of skill can produce short nucleic acid sequences using chemical synthesis and long nucleic acid sequences using cloning, mutagenesis, or polymerase chain reactions. Unfortunately, although the design and production of DNA is common, the process currently takes a high level of skill, and the manufacture of accurate DNA constructs is severely impacted by error rates inherent in the commonly used chemical synthesis techniques. For example, the synthesis of a DNA having an open reading frame of 3 kb using a method with an error rate of 1 base in 1000 bases will result in less than 5% of the copies of the synthesized DNA having the correct sequence.

The use of oligonucleotides to create assembly products for the production of polynucleotides creates a reliance on the fidelity of the assembly product. Since a low fidelity assembly product creates a high error rate in the production of the polynucleotide, the creation of a high fidelity assembly product is desired to reduce the error rate and obtain a more accurate polynucleotide product at a higher yield than what would be realized from corresponding low fidelity product. Since current methods for generating even the simplest of oligonucleotides are expensive and have high error rates, methods that are less expensive and less prone to such error will be received well by those of skill.

Currently, there are several hurdles that exist in the production of a desired polynucleotide including: (1) a high level of skill is required to design a desired polynucleotide, preselect a set of oligonucleotides to build the assembly product, and determine the reaction conditions necessary to assemble the assembly product; (2) the reagents used to produce the desired polynucleotids are expensive; and (3) the delays inherent in current processing of an order for the desired polynucleotide or reagents create research and development bottlenecks. Although these hurdles impede research and development, the ultimate price of these hurdles is paid by the consumer, who suffers in that any innovations can come slow and at a high cost.

Accordingly, the field of polynucleotide synthesis would benefit from a kit, system, or method that enables a user having a low level of skill in the art to design a desired polynucleotide, preselect and order a set of custom oligonucleotides that can complement the design, and quickly assemble a high fidelity assembly product. The kit, systems, and methods taught herein, however, are even more robust in that they are also equipped to enable a user having a high level of skill in the art of gene construction and synthesis to modify a polynucleotide according to the numerous design elements and features familiar to such persons.

### SUMMARY

This disclosure is directed to the field of polynucleotide synthesis, and the embodiments taught herein are generally directed to a kit for use in the design of a desired polynucleotide from information obtained from a polypeptide or another polynucleotide, the generation of a custom set of oligonucleotides that complement the design, and the ordering of the custom set of oligonucleotides from an outside provider of oligonucleotides. The invention includes systems and methods for producing a desired polynucleotide using the kit.

In some embodiments, the disclosure is directed to a custom synthesis kit for producing a desired polynucleotide, wherein the kit comprises a computer program for use by a developer. The developer designs a desired polynucleotide from a first polynucleotide or a first polypeptide and preselects a custom set of oligonucleotides that will assemble to create an assembly product for producing the desired polynucleotide. The skill of the developer ranges from a low level to a high level in the art of polynucleotide synthesis, and is not a provider of oligonucleotides or affiliated with such a provider. In these embodiments, there can also be a means for ordering the custom set of oligonucleotides from an outside source. In some embodiments, the assembly product is a high-fidelity assembly product, such that at least 25% of the assembly product produces the desired polynucleotide.

In some embodiments, the designing includes entering sequence information from the first polypeptide or the first polynucleotide into the first component to generate information selected from a group consisting of repetitive elements, inverted repeats, GC content, restriction sites, stop codons and multiple frames, CPG motifs, methylation patterns, and combinations thereof, about the desired polynucleotide used in the preselecting of the set of custom oligonucleotides. And, in some embodiments, the kit further comprises a set of custom reaction conditions, wherein the set of custom synthesis reaction conditions are in the form of a digital display, a printout, and/or a computer file or program used to instruct a thermocycler to implement the set of custom synthesis reaction conditions. In many embodiments, however, the kit is designed for use by persons having a low level of skill in the art of polynucleotide synthesis. The desired polynucleotide can also be produced using a single-pot assembly of the assembly product.

The disclosure also teaches embodiments directed to a system for producing a desired polynucleotide using the custom synthesis kit. The system comprises a first component comprising the kit, and a second component designed by the developer to specifically complement the first component. The second component comprises the set of custom oligonucleotides, a set of custom reagents, and a set of custom synthesis reaction conditions for denaturing annealing, and extension, to produce the assembly product using a thermocycler. In these embodiments, the designing includes entering sequence information from the first polypeptide or the first polynucleotide into the first component to generate information about the desired polynucleotide used in the preselecting of the set of custom oligonucleotides; and, the developer is not a provider of the second component or affiliated with such a provider. In some embodiments, the designing of the desired polynucleotide and preselecting of the set of oligonucleotides can result in the formation of a high-fidelity assembly product, such that at least 25% of the assembly product produces the desired polynucleotide.

In some embodiments, the system further comprises a set of custom reaction conditions, wherein the set of custom synthesis reaction conditions are in the form of a digital display, a printout, and/or a computer file or program used to instruct a thermocycler to implement the set of custom synthesis reaction conditions. In many embodiments, wherein the kit is designed for use by persons having a low level of skill in the art of polynucleotide synthesis. In some embodiments, the desired polynucleotide is produced using a single-pot assembly of the assembly product.

The invention includes embodiments directed to method of producing a polynucleotide with the custom synthesis kit. The method includes using the kit for the designing of the desired polynucleotide from the first polynucleotide or the first polypeptide and the preselecting of the set of custom oligonucleotides. In these embodiments, the designing includes entering sequence information from the first polypeptide or the first polynucleotide into the computer program to generate information about the desired polynucleotide, ordering the custom set of oligonucleotides from an outside source, and producing the desired polynucleotide with a thermocycler. In some embodiments, the designing includes selecting a modification to the first polynucleotide or first polypeptide, and the modification is selected from a group consisting of a point mutation, a variant, a chimeric construction, a codon bias of a host cell, a sequence length, and a combination thereof, such that the desired polynucleotide provides a specific expression system. In many embodiments, the kit is designed for use by persons having a low level of skill in the art of polynucleotide synthesis.

### BRIEF DESCRIPTION OF THE FIGURES

FIG. 1 illustrates a polynucleotide synthesis system according to some embodiments of the present invention.

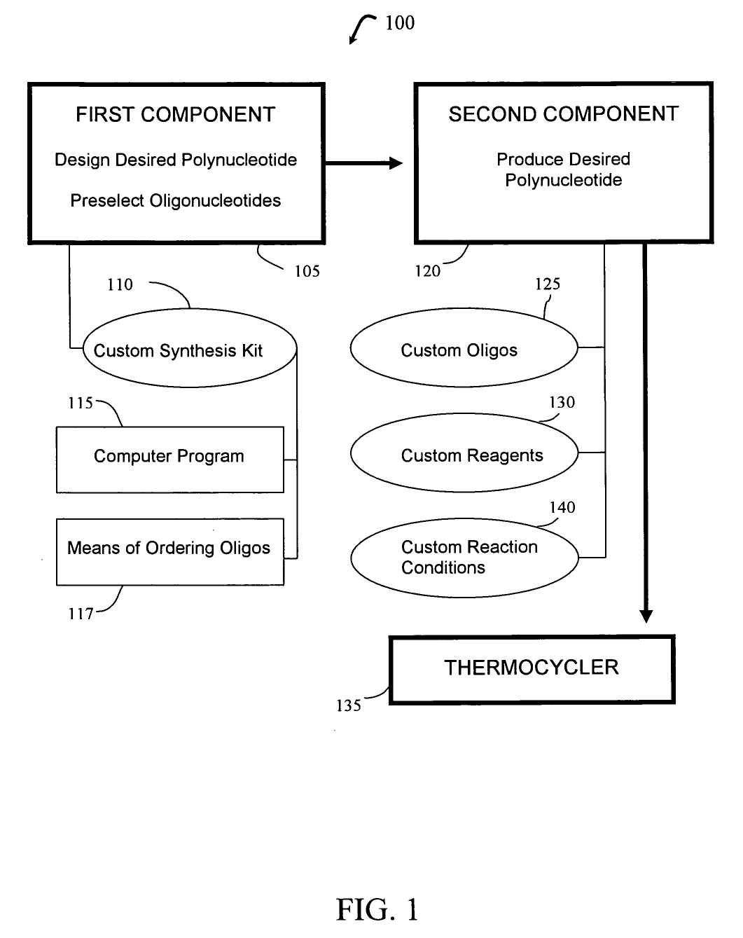

FIG. 2 illustrates a method of producing a polynucleotide according to some embodiments of the present invention.

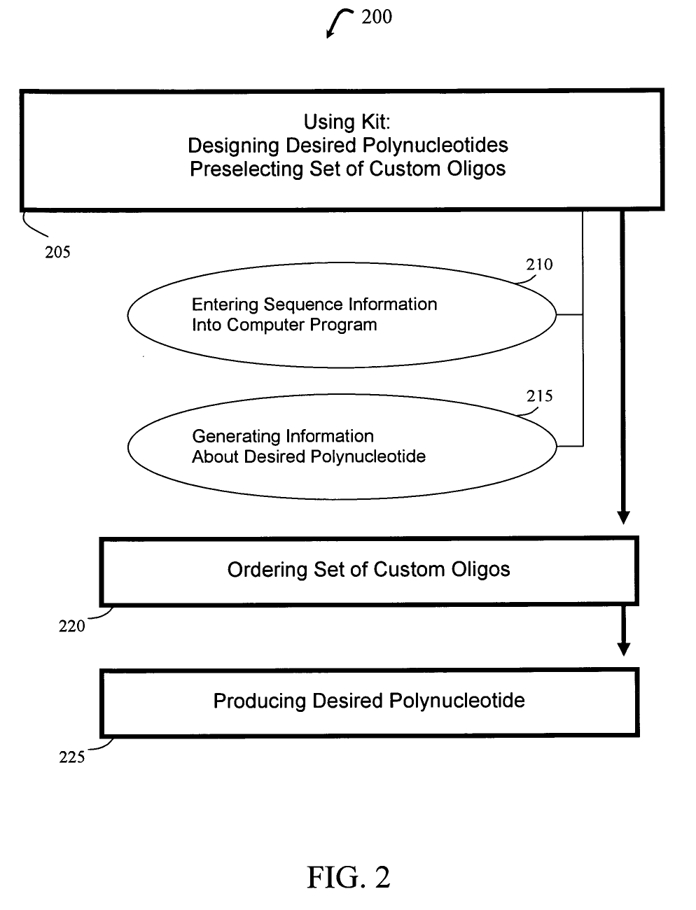

### DETAILED DESCRIPTION

The embodiments taught herein are generally directed to a kit for use in the design of a desired polynucleotide from information obtained from a polypeptide or polynucleotide, the generation of a custom set of oligonucleotides that complement the design, and the ordering of the custom set of oligonucleotides from an outside provider of oligonucleotides. The invention can also comprise systems and methods for producing a desired polynucleotide using the kit.

In many embodiments, the invention can include a custom synthesis kit for producing a desired polynucleotide, wherein the kit comprises a computer program for use by a developer. The computer programs used in the embodiments taught herein can be any program for designing a desired polynucleotide from a first polynucleotide or a first polypeptide, and preselecting a complementary custom set of oligonucleotides that will assemble to create an assembly product for producing the desired polynucleotide, wherein the skill level of the developer can range from a low level of skill to a high level of skill in the art of polynucleotide synthesis. Although a computer program that is immediately suitable for the present invention can be obtained from Gene Oracle, 922 San Leandro Ave, Mountain View, Calif. 94043, other programs may be suitable for alteration to make them suitable for use by those having a low level skill in the art. These programs include, but are not limited to, DNAWorks, available at http://helixweb.nih.gov/dnaworks/; and Gene2Oligos, available at http://berry.engin.umich.edu/gene2oligo/. Regardless of whether the program is obtained directly from Gene Oracle, or obtained from another source and altered, the program should require simple input information to design a desired polynucleotide, such that the input is so simple that it can be provided by a person having a low level of skill in the art of polynucleotide synthesis. And, the program should readily provide an output containing a custom set of oligonucleotides that complement the polynucleotide design and produce a high quality assembly product.

In some embodiments, the assembly product formed from the design and selection is an assembly product, such that at least 10%, 15%, 20%, 25%, 35%, 40%, 45%, 50%, or any range therein, of the assembly product produces the desired polynucleotide. In some embodiments, the assembly product is a high-fidelity assembly product having at least 25-50%, or any range therein, of the assembly product producing the desired polynucleotide.

A first polynucleotide or first polypeptide can include, but is not limited to any wild-type sequence, recombinant sequence, synthetic sequence, and the like, used by a developer as a basis upon which to begin developing a desired polynucleotide. In some embodiments, the desired polynucleotide can be used as part of an expression system to produce a desired protein. In some embodiments, the desired polynucleotide can be used to interfere with the expression of an undesired protein. In many embodiments, designing the desired polynucleotide includes entering sequence information from the first polypeptide or the first polynucleotide into the computer program to generate information selected from a group consisting of repetitive elements, inverted repeats, GC content, restriction sites, stop codons and multiple frames, CPG motifs, methylation patterns, and combinations thereof, about the desired polynucleotide.

The generated information is used by the computer program for preselecting the set of custom oligonucleotides—the oligonucleotides are custom because they are preselected according to the design of the desired polynucleotide to form an assembly product corresponding to the desired polynucleotide. In some embodiments, the computer generates a set of custom reaction conditions that further complement the assembly of the set of custom oligonucleotides. In some embodiments, this set of custom synthesis reaction conditions can include, for example, the temperature, time-at-temperature, and number of cycles for the denaturing, annealing, and extension. Any combination of time, temperature, and number of cycles can be generated and repeated for the custom reaction conditions. For example, in some embodiments, the custom reaction conditions can include a singe cycle of denaturing, annealing, and extension, whereas in other embodiments, there can be several cycles, and the conditions can either vary or repeat during the cycling. These custom reaction conditions can be generated in the form of a digital display, a printout, and/or a computer file or program, each of which can be used to instruct a thermocycler to implement the set of custom synthesis reaction conditions.

In most embodiments, the developer is not a provider of oligonucleotides or affiliated with such a provider. The custom synthesis kit can, for example, allow the developer to design a desired polynucleotide, preselect a custom set of oligonucleotides, and directly order those oligonucleotides from such a provider, without ever having to wait for the same or different service provider to also design the desired polynucleotide and preselect the oligonucleotides for the assembly product. In many embodiments, the provider is an “outside source,” such that the developer is neither the provider of the oligonucleotides nor a business affiliate of the provider. A business affiliate, in some embodiments, would include a business concern that is subject to common operating control and/or operated as part of the same system or enterprise. Although one of skill can readily obtain the set of custom oligonucleotides from several additional sources, such outside providers can include Integrated DNA Technologies Inc., 1710 Commercial Park, Coralville, Iowa 52241; Invitrogen Inc., 1600 Faraday Ave. PO Box 6482, Carlsbad, Calif. 92008; Sigma-Genosys Company, The Woodlands, Tex.; and Operon Biotechnologies, Inc., 2211 Seminole Drive, Huntsville, Ala. 35805.

For example, the developer may be a researcher in a laboratory, either academic or commercial, having a thermocycler and the basic skills necessary to create the assembly product and produce the desired polynucleotide. The developer designs a desired polynucleotide from a first polynucleotide or a first polypeptide, preselects a custom set of oligonucleotides that will assemble to create an assembly product for producing the desired polynucleotide, and places an order for the custom set of oligonucleotides from the outside source. Accordingly, in these embodiments, the researcher's delay in proceeding with the polynucleotide synthesis can be limited to one outside source—the oligonucleotide provider.

The skill of the developer can range from a low level to a high level in the art of polynucleotide synthesis, and is not a provider of oligonucleotides or affiliated with such a provider. A person having a low level skill may include, for example, a person having a minimal skillset in the field and capable of being instructed on how to enter information on the first polynucleotide or first polypeptide into the computer program. In some embodiments, the entry of the information merely comprises entering a computer file containing that information as an input to the computer program. A person having a high level of skill may include, for example, a person having a thorough understanding of the art of gene construction and synthesis, as well as the effects expected from modifications to the genes, oligonucleotides, reagents, and reaction conditions.

The means for ordering the custom set of oligonucleotides from the outside source can be any means for transmitting the information about the custom set of oligonucleotides to the outside provider of oligonucleotides, whether electronic or hardcopy. The means for ordering can include, but is not limited to, transmission of a computer printout by electronic or regular mail; transmission using a telephone number, such as a facsimile transmission; transmission through an electronic a link between the computer program and the outside provider, such as through an internet connection; and the like.

In some embodiments, the designing includes entering sequence information from the first polypeptide or the first polynucleotide into a first component containing the computer program to generate information that can be selected from a group consisting of repetitive elements, inverted repeats, GC content, restriction sites, stop codons and multiple frames, CPG motifs, methylation patterns, and combinations thereof, about the desired polynucleotide used in the preselecting of the set of custom oligonucleotides. And, in some embodiments, the kit can further comprise the set of custom reaction conditions, wherein the set of custom synthesis reaction conditions may be in the form of a digital display, a printout, and/or a computer file or program used to instruct a thermocycler to implement the set of custom synthesis reaction conditions. In many embodiments, however, the kit can be designed for use by persons having a low level of skill in the art of polynucleotide synthesis. The desired polynucleotide can also be produced using assembly methods that include a single-pot assembly of the assembly product. However, in some embodiments, the assembly can comprise a multiple-pot assembly.

The invention includes embodiments directed to a system 100 for producing a desired polynucleotide using the custom synthesis kit. The system comprises a first component 105 comprising the kit 110 having the computer program 115 and the means 117 for ordering the custom set of oligonucleotides, and a second component 120 designed by the developer to specifically complement the first component 105.

The first component 105 is used to design the desired polynucleotide from the first polynucleotide or the first polypeptide and preselect the custom set of oligonucleotides that will assemble to create the assembly product for producing the desired polynucleotide. The second component 120 is designed by the developer to specifically complement the first component 105 and can comprise the set of custom oligonucleotides 125, and a set of custom reagents 130 to produce the assembly product using a thermocycler 135. The second component 120 is designed by the developer to specifically complement the first component. The set of custom reagents 130 designed by the developer should contain the reagents necessary for production of the desired polynucleotide. One of skill will appreciate that such reagents will often include, but are not limited to, components selected from a group consisting of a salt solution (magnesium, potassium, or sodium salts as a chloride or sulfate); bovine serum albumin (BSA); buffer (phosphate buffer, tris buffer); dimethylsulfoxide; deoxyribonucleotides; enzymes (polymerase, ligase); and premixed enzymes (polymerase or ligase premixed in a buffer).

In some embodiments, the system 100 further comprises a set of custom reaction conditions 140 for the synthesis steps of denaturing, annealing, and extension, wherein the set of custom synthesis reaction conditions are in the form of a digital display, a printout, and/or a computer'file or program used to instruct a thermocycler 135 in performing the reaction. In many embodiments, the system 100 can be designed for use by persons having a low level of skill in the art of polynucleotide synthesis. In some embodiments, the desired polynucleotide is produced by the system 100 using methods that include a single-pot assembly of the assembly product. However, in some embodiments, the assembly comprises a multiple-pot assembly.

The invention includes embodiments directed to method of producing a polynucleotide with the custom synthesis kit. The method includes using 205 the kit for the designing of the desired polynucleotide from the first polynucleotide or the first polypeptide and the preselecting of the set of custom oligonucleotides. In these embodiments, the designing includes entering 210 sequence information from the first polypeptide or the first polynucleotide into the computer program to generate 215 information about the desired polynucleotide used in the preselecting of the set of custom oligonucleotides, ordering 220 the custom set of oligonucleotides from an outside source, and producing 225 the desired polynucleotide with a thermocycler. In some embodiments, the developer is not a provider of the second component or affiliated with such a provider.

In some embodiments, the designing includes selecting a modification to the first polynucleotide or first polypeptide, and the modification can be selected from a group consisting of a point mutation, a variant, a chimeric construction, a codon bias of a host cell, a sequence length, and a combination thereof, such that the desired polynucleotide provides a specific expression system. In many embodiments, the kit is designed for use by persons having a low level of skill in the art of polynucleotide synthesis.

The polynucleotides of the present invention can be produced to a variety of different sequence lengths, and the sequence length that can be obtained can be limited by whether the assembly method is a single-pot assembly or a multiple-pot assembly. In some embodiments, the sequence length can be up to about 7 kb. In some embodiments, the sequence length can be up to about 2 kb. In some embodiments, the sequence length can range from up to about 0.01-2 kb, 1-3 kb, 2-5 kb, 3-5 kb, 5-7 kb, or any range therein.

One of skill will recognize that the following examples are very limited and are intended only to illustrate a few embodiments of the present invention; as such, it should be appreciated that these examples are not intended to limit the invention in any way.

### Example 1

In this example, a developer wants to produce an insulin-encoding nucleic acid based on the following sequence.

(SEQ ID NO:1)

5′gcattctgaggcattctctaacaggttctcgaccctccgccatggccc
cgtggatgcatctcctcaccgtgctggccctgctggccctctggggaccc
aactctgttcaggcctattccagccagcacctgtgcggctccaacctagt
ggaggcactgtacatgacatgtggacggagtggcttctatagaccccacg
accgccgagagctggaggacctccaggtggagcaggcagaactgggtctg
gaggcaggcggcctgcagccttcggccctggagatgattctgcagaagcg
cggcattgtggatcagtgctgtaataacatttgcacatttaaccagctgc
agaactactgcaatgtcccttagacacctgccttgggcctggcctgctgc
tctgccctggcaaccaataaaccccttgaatgag 3′

Based on the above sequence, the developer uses the first component of the system to design the desired polynucleotide and preselect the following set of custom oligonucleotides arranged in Table 1 in a 96 well ordering format:

TABLE 1

| | | | | |
|---|---|---|---|---|
| | | | | SEQ ID |
| Well | Oligo | Sequence (5′ to 3′) | length | NO. |

| | | | | |
|---|---|---|---|---|
| A1 | Insulin AF | Agcattctgaggcattctctaacag primer | 25 | 2 |
| B1 | Insulin AR | Agagtaagttccccaaataaccaacg primer | 26 | 3 |
| A2 | Insulin F1 | gcattctgaggcattctctaacaggt | 26 | 4 |
| B2 | Insulin R1 | cgcctcccagctcttggacaatctcttacgg | 31 | 5 |
| C2 | Insulin F2 | tctcgaccctccgccatggccccgtgg | 27 | 6 |
| D2 | Insulin R2 | gccactcctctacgtaggtgccccggtac | 29 | 7 |
| E2 | Insulin F3 | atgcatctcctcaccgtgctggccctgctg | 30 | 8 |
| F2 | Insulin R3 | ccaggggtctcccggtcgtcccggtcgt | 28 | 9 |
| G2 | Insulin F4 | gccctctggggacccaactctgttcaggcc | 30 | 10 |
| H2 | Insulin R4 | ccacgaccgaccttatccggacttgtctcaac | 32 | 11 |
| A3 | Insulin F5 | tattccagccagcacctgtgcggctccaac | 30 | 12 |
| B3 | lnsulin R5 | tgtcacggaggtgatccaacctcggcgtgt | 30 | 13 |
| C3 | Insulin F6 | ctagtggaggcactgtacatgacatgtggacgg | 33 | 14 |
| D3 | Insulin R6 | cccagatatcttcggtgaggcaggtgtacagtaca | 35 | 15 |
| E3 | Insulin F7 | agtggcttctatagaccccacgaccgccgag | 31 | 16 |
| F3 | Insulin R7 | cctccaggaggtcgagagccgccagcac | 28 | 17 |
| G3 | Insulin F8 | agctggaggacctccaggtggagcaggc | 28 | 18 |
| H3 | Insulin R8 | gaggtctgggtcaagacggacgaggtgga | 29 | 19 |
| A4 | Insulin F9 | agaactgggtctggaggcaggcggcctg | 28 | 20 |
| B4 | Insulin R9 | cccggcttccgacgtccggcggacg | 25 | 21 |
| C4 | Insulin F10 | cagccttcggccctggagatgattctgcagaa | 32 | 22 |
| D4 | Insulin R10 | gtgttacggcgcgaagacgtcttagtagaggt | 32 | 23 |
| E4 | Insulin F11 | gcgcggcattgtggatcagtgctgtaataacat | 33 | 24 |
| F4 | Insulin R11 | tcgaccaatttacacgtttacaataatgtcgtgactag | 38 | 25 |
| G4 | Insulin F12 | ttgcacatttaaccagctgcagaactactgcaatg | 35 | 26 |
| H4 | Insulin R12 | ccgtccacagattccctgtaacgtcatcaagacg | 34 | 27 |
| A5 | Insulin F13 | tcccttagacacctgccttgggcctggcct | 30 | 28 |
| B5 | Insulin R13 | tcccgtctcgtcgtccggtccgggtt | 26 | 29 |
| C5 | Insulin F14 | gctgctctgccctggcaaccaataaacccc | 30 | 30 |
| D5 | Insulin R14 | gagtaagttccccaaataaccaacgg | 26 | 31 |

Knowing the content of the set of custom oligonucleotides, the developer can then simply order the custom set of oligonucleotides from an outside provider of oligonucleotides. Upon receiving the custom set of oligonucleotides, the developer can create the second component containing the custom set of oligonucleotides, a set of custom reagents, and a set of custom reaction conditions, since information regarding each of which can be generated by the first component. Using the second component, the developer mixes the oligonucleotides Insulin F1 through Insulin R14 to reach a final concentration of 10-50 μM. The developer then makes up a reaction mixture with a concentration of sodium salt of 250 to 500 mM and a concentration of DMSO of 0 to 5% and follows the custom reaction conditions. The set of custom reaction conditions can differ, depending on the design selected by the developer.

According to one set of custom reaction conditions, the developer can subject the reaction mixture to the following thermal cycling program:

| | |
|---|---|
| Denature | 95° C. for 30 seconds |
| Anneal | 72° C. for 25 seconds |
| Extend | 55° C. for 30 seconds |
| No. Cycles | 20 Cycles |

According to another set of custom reaction conditions, the user can subject the reaction mixture to the following thermal cycling program:

| | |
|---|---|
| Denature | 94.0° C. for 1 second |
| Anneal | 60.9° C. for 30 seconds |
| Extend | 72.0° C. for 8 seconds |
| No. Cycles | 1 cycle |
| Denature | 94.0° C. for 1 second |
| Anneal | 60.1° C. for 30 seconds |
| Extend | 72.0° C. for 10 seconds |
| No. Cycles | 4 cycles |
| Denature | 94.0° C. for 1 second |
| Anneal | 59.3° C. for 30 seconds |
| Extend | 72.0° C. for 12 seconds |
| No. Cycles | 4 cycles |
| Denature | 94.0° C. for 1 second |
| Anneal | 58.5° C. for 30 seconds |
| Extend | 72.0° C. for 12 seconds |
| No. Cycles | 4 cycles |
| Denature | 94.0° C. for 1 second |
| Anneal | 57.7° C. for 30 seconds |
| Extend | 72.0° C. for 16 seconds |
| No. Cycles | 4 cycles |
| Denature | 94.0° C. for 1 second |
| Anneal | 56.9° C. for 30 seconds |
| Extend | 72.0° C. for 16 seconds |
| No. Cycles | 1 cycle |

### Example 2

A developer intends to synthesize a Green Fluorscent Protein (GFP)-encoding nucleic acid of the following sequence:

(SEQ ID NO:32)

5′atgagtaaaggagaagaacttttcactggagttgtcccaattcttgtt
gaattagatggtgatgttaatgggcacaaattttctgtcagtggagaggg
tgaaggtgatgcaacatacggaaaacttacccttaaatttatttgcacta
ctggaaaactacctgttccatggccaacacttgtcactactttcggttat
ggtgttcaatgctttgcgagatacccagatcatatgaaacagcatgactt
tttcaagagtgccatgcccgaaggttatgtacaggaaagaactatatttt
tcaaagatgacgggaactacaagacacgtgctgaagtcaagtttgaaggt
gatacccttgttaatagaatcgagttaaaaggtattgattttaaagaaga
tggaaacattcttggacacaaattggaatacaactataactcacacaatg
tatacatcatggcagacaaacaaaagaatggaatcaaagttaacttcaaa
attagacacaacattgaagatggaagcgttcaactagcagaccattatca
acaaaatactccaattggcgatggccctgtccttttaccagacaaccatt
acctgtccacacaatctgccctttcgaaagatcccaacgaaaagagagac
cacatggtccttcttgagtttgtaacagctgctgggattacacatggcat
ggatgaactatacaaatag 3′

Based on the above sequence, the developer uses the first component to design the desired nucleotide and preselect the custom set of oligonucleotides arranged in Table 2 in a 96 well ordering format:

TABLE 2

| | | | | SEQ. ID |
|---|---|---|---|---|
| Well | Oligo | Sequence (5′ to 3′) | length | NO. |
| A1 | GFPAF | Aatgagtaaaggagaagaacttttcact primer | 28 | 33 |
| B1 | GEPAR | Agataaacatatcaagtaggtacggtacac primer | 30 | 34 |
| C1 | SeqF1 | Actacaagacacgtgctgaa primer | 20 | 35 |
| D1 | SegR1 | Cacaggttcttacaaaggtagaaga primer | 25 | 36 |
| A2 | GFPF1 | atgagtaaaggagaagaacttttcactg | 28 | 37 |
| B2 | GFPR1 | gttcttaaccctgttgaggtcacttttcaagaagagg | 37 | 38 |
| C2 | GFPF2 | gagttgtcccaattcttgttgaattagatggtgatgttaat | 41 | 39 |
| D2 | GFPR2 | tgtcttttaaacacgggtaattgtagtggtagattaagtt | 40 | 40 |
| E2 | GFPF3 | gggcacaaattttctgtcagtggagagggtga | 32 | 41 |
| F2 | GFPR3 | gcatacaacgtagtggaagtgggagaggtgac | 32 | 42 |
| G2 | GFPF4 | aggtgatgcaacatacggaaaacttacccttaaatttattt 41 | 43 | |
| H2 | GFPR4 | catcaaaaggtcatcacgtttatttaaattcccattcaaaag | 42 | 44 |
| A3 | GFPF5 | gcactactggaaaactacctgttccatggccaac | 34 | 45 |
| B3 | GFPR5 | ggctttcatcactgttcacaaccggtaccttgtc | 34 | 46 |
| C3 | GFPF6 | acttgtcactactttcggttatggtgttcaatgctttg | 38 | 47 |
| D3 | GFPR6 | atactagacccatagagcgtttcgtaacttgtggtatt | 38 | 48 |
| E3 | GFPF7 | cgagatacccagatcatatgaaacagcatgactttt | 36 | 49 |
| F3 | GFPR7 | ccgtaccgtgagaactttttcagtacgacaaagt | 34 | 50 |
| G3 | GFPF8 | tcaagagtgccatgcccgaaggttatgtacaggaa | 35 | 51 |
| H3 | GFPR8 | cagtagaaactttttatatcaagaaaggacatgtattggaagc | 43 | 52 |
| A4 | GFPF9 | agaactatatttttcaaagatgacgggaactacaagacacg | 41 | 53 |
| B4 | GFPR9 | gaagtttgaactgaagtcgtgcacagaacatcaaggg | 37 | 54 |
| C4 | GEPF10 | tgctgaagtcaagtttgaaggtgatacccttgttaatagaa | 41 | 55 |
| D4 | GEPR10 | tagttatggaaaattgagctaagataattgttcccatagtg | 41 | 56 |
| E4 | GEPF11 | tcgagttaaaaggtattgattttaaagaagatggaaacattc | 42 | 57 |
| F4 | GEPR11 | cataaggttaaacacaggttcttacaaaggtagaagaaattt | 42 | 58 |
| G4 | GFPF12 | ttggacacaaattggaatacaactataactcacacaatgt | 40 | 59 |
| H4 | GFPR12 | aacagacggtactacatatgtaacacactcaatatcaa | 38 | 60 |
| A5 | GEPE13 | atacatcatggcagacaaacaaaagaatggaatcaaag | 38 | 61 |
| B5 | GEPR13 | acaacacagattaaaacttcaattgaaactaaggtaagaaaaca | 44 | 62 |
| C5 | GFPF14 | ttaacttcaaaattagacacaacattgaagatggaagcgt | 40 | 63 |
| D5 | GEPR14 | tattaccagacgatcaacttgcgaaggtagaagtt | 35 | 64 |
| E5 | GFPF15 | tcaactagcagaccattatcaacaaaatactccaattgg 39 | 65 | |
| F5 | GEPR15 | cctgtcccggtagcggttaacctcataaaacaac | 34 | 66 |
| G5 | GEPE16 | cgatggccctgtccttttaccagacaaccattac | 34 | 67 |
| H5 | GEPR16 | cgtctaacacacctgtccattaccaacagaccatttt | 37 | 68 |
| A6 | GFPF16 | ctgtccacacaatctgccctttcgaaagatccca | 34 | 69 |
| B6 | GEPR17 | caccagagagaaaagcaaccctagaaagctttcc | 34 | 70 |
| C6 | GFPF18 | acgaaaagagagaccacatggtccttcttgagtt | 34 | 71 |
| D6 | GFPR18 | gggtcgtcgacaatgtttgagttcttcctggta | 33 | 72 |
| E6 | GFPF19 | tgtaacagctgctgggattacacatggcatgga | 33 | 73 |
| F6 | GEPR19 | gataaacatatcaagtaggtacggtacacatta | 33 | 74 |

The developer orders the custom set of oligonucleotides from an outside provider of oligonucleotides, and creates the second component after receiving the custom set of oligonucleotides, which contains the custom set of oligonucleotides, a custom set of reagents, and a custom set of reaction conditions. The developer then mixes the oligonucleotides GFPF1 through GFPR19 to a final concentration of 10-50 μM, creates a reaction mixture with a concentration of sodium salt of 250 to 500 mM and a concentration of DMSO 0 to 5%, and subjects the reaction mixture to the following thermal cycling program according to the custom set of reaction conditions:

| | |
|---|---|
| Step 1 | 95° C. for 30 seconds |
| Step 2 | 72° C. for 45 seconds |
| Step 3 | 55° C. for 30 seconds |
| Duration | 25 Cycles |

### Example 3

A user intends to synthesize a Tetracycline Resistance gene (tetR)-encoding nucleic acid of the following sequence.

(SEQ ID NO:75)

5′ATGAATAGTTCGACAAAGATCGCATTGGTAATTACGTTACTCGATGCC
ATGGGGATTGGCCTTATCATGCCAGTCTTGCCAACGTTATTACGTGAATT
TATTGCTTCGGAAGATATCGCTAACCACTTTGGCGTATTGCTTGCACTTT
ATGCGTTAATGCAGGTTATCTTTGCTCCTTGGCTTGGAAAAATGTCTGAC
CGATTTGGTCGGCGCCCAGTGCTGTTGTTGTCATTAATAGGCGCATCGCT
GGATTACTTATTGCTGGCTTTTTCAAGTGCGCTTTGGATGCTGTATTTAG
GCCGTTTGCTTTGAGGGATCACAGGAGCTACTGGGGCTGTCGCGGCATCG
GTCATTGCCGATACCACCTCAGCTTCTCAACGCGTGAAGTGGTTCGGTTG
GTTAGGGGCAAGTTTTGGGCTTGGTTTAATAGCGGGGCCTATTATTGGTG
GTTTTGCAGGAGAGATTTCACCGCATAGTCCCTTTTTTATCGCTGCGTTG
CTAAATATTGTCACTTTCCTTGTGGTTATGTTTTGGTTCCGTGAAACCAA
AAATACACGTGATAATACAGATACCGAAGTAGGGGTTGAGACGCAATCGA
ATTCGGTATACATCACTTTATTTAAAACGATGCCCATTTTGTTGATTATT
TATTTTTCAGCGCAATTGATAGGCCAAATTCCCGCAACGGTGTGGGTGCT
ATTTACCGAAAATCGTTTTGGATGGAATAGCATGATGGTTGGCTTTTCAT
TAGCGGGTCTTGGTCTTTTACACTCAGTATTCCAAGCCTTTGTGGCAGGA
AGAATAGCCACTAAATGGGGCGAAAAAACGGCAGTACTGCTCGAATTTAT
TGCAGATAGTAGTGCATTTGCCTTTTTAGCGTTTATATCTGAAGGTTGGT
TAGATTTCCCTGTTTTAATTTTATTGGCTGGTGGTGGGATCGCTTTACCT
GCATTACAGGGAGTGATGTCTATCCAAACAAAGAGTCATGAGCAAGGTGC
TTTACAGGGATTATTGGTGAGCCTTA 3′

Based on the above sequence, the developer designs and preselects the following oligonucleotides, arranged below in Table 3 in a 96 well ordering format:

TABLE 3

| | | | | |
|---|---|---|---|---|
| | | | | SEQ ID |
| Well | Oligo | Sequence (5′ to 3′) | length | NO. |

| | | | | |
|---|---|---|---|---|
| A1 | Tet | AATGAATAGTTCGACAAAGATCGCA | 25 | 76 |
| | RAF | | | |
| B1 | Tet | ACTAAGCACTTGTCTCCTGTTTACT | 25 | 77 |
| | RAR | | | |
| C1 | SeqF1 | ACACGTGATAATACAGATACCGAAG | 25 | 78 |
| A2 | Tet | ATGAATAGTTCGACAAAGATCGCATTGGTAATTAC | 35 | 79 |
| | RF1 | | | |
| B2 | Tet | CCCATGGCATCGAGTAACGTAATTACCAATGCGATCTTTG | 40 | 80 |
| | RR1 | | | |
| C2 | Tet | GTTACTCGATGCCATGGGGATTGGCCTTATCATGCC | 36 | 81 |
| | RF2 | | | |
| D2 | Tet | GTAATAACGTTGGCAAGACTGGCATGATAAGGCCAATC | 38 | 82 |
| | RR2 | | | |
| E2 | Tet | AGTCTTGCCAACGTTATTACGTGAATTTATTGCTTCGGAAG | 41 | 83 |
| | RF3 | | | |
| F2 | Tet | CCAAAGTGGTTAGCGATATCTTCCGAAGCAATAAATTCAC | 40 | 84 |
| | RR3 | | | |
| G2 | Tet | ATATCGCTAACCACTTTGGCGTATTGCTTGCACTTTATG | 39 | 85 |
| | RF4 | | | |
| H2 | Tet | CAAAGATAACCTGCATTAACGCATAAAGTGCAAGCAATACG | 41 | 86 |
| | RR4 | | | |
| A3 | Tet | CGTTAATGCAGGTTATCTTTGCTCCTTGGCTTGGAAAAA | 39 | 87 |
| | RF5 | | | |
| B3 | Tet | ACCAAATCGGTCAGACAT1TVTCCAAGCCAAGGAG | 35 | 88 |
| | RR5 | | | |
| C3 | Tet | TGTCTGACCGATTTGGTCGGCGCCCAGTG | 29 | 89 |
| | RF6 | | | |
| D3 | Tet | CGCCTATTAATGACAACAACAGCACTGGGCGCCG | 34 | 90 |
| | RR6 | | | |
| E3 | Tet | CTGTTGTTGTCATTAATAGGCGCATCGCTGGATTACTTATTGC | 43 | 91 |
| | RF7 | | | |
| F3 | Tet | GCGCACTTGAAAAAGCCAGCAATAAGTAATCCAGCGATG | 39 | 92 |
| | RR7 | | | |
| G3 | Tet | TGGCTTTTTCAAGTGCGCTTTGGATGCTGTATTTAGGCC | 39 | 93 |
| | RF8 | | | |
| H3 | Tet | GTGATCCCTGAAAGCAAACGGCCTAAATACAGCATCCAAA | 40 | 94 |
| | RR8 | | | |
| A4 | Tet | GTTTGCTTTCAGGGATCACAGGAGCTACTGGGGC | 34 | 95 |
| | RF9 | | | |
| B4 | Tet | CCGATGCCGCGACAGCCCCAGTAGCTCCT | 29 | 96 |
| | RR9 | | | |
| C4 | Tet | TGTCGCGGCATCGGTCATTGCCGATACCACCT | 32 | 97 |
| | RF10 | | | |
| D4 | Tet | CGCGTTGAGAAGCTGAGGTGGTATCGGCAATGA | 33 | 98 |
| | RR10 | | | |
| E4 | Tet | CAGCTTCTCAACGCGTGAAGTGGTTCGGTTGGT | 33 | 99 |
| | RF11 | | | |
| F4 | Tet | CCCAAAACTTGCCCCTAACCAACCGAACCACTTCA | 35 | 100 |
| | RR11 | | | |
| G4 | Tet | TAGGGGCAAGTTTTGGGCTTGGTTTAATAGCGGGG | 35 | 101 |
| | RF12 | | | |
| H4 | Tet | TGCAAAACCACCAATAATAGGCCCCGCTATTAAACCAAG | 39 | 102 |
| | RR12 | | | |
| A5 | Tet | CCTATTATTGGTGGTTTTGCAGGAGAGATTTCACCGCA | 38 | 103 |
| | RF13 | | | |
| B5 | Tet | GAGCGATAAAAAAGGGACTATGCGGTGAAATCTCTCC | 37 | 104 |
| | RR13 | | | |
| C5 | Tet | TAGTCCCTTTTTTATCGCTGCGTTGCTAAATATTGTCACTT | 41 | 105 |
| | RF14 | | | |
| D5 | Tet | CCAAAACATAACCACAAGGAAAGTGACAATATTTAGCAACG | 41 | 106 |
| | RR14 | | | |
| E5 | Tet | TCCTTGTGGTTATGTTTTGGTTCCGTGAAACCAAAAATACAC | 42 | 107 |
| | RF15 | | | |
| F5 | Tet | CTACTTCGGTATCTGTATTATCACGTGTATTTTTGGTTTCACGGAA | 46 | 108 |
| | RR15 | | | |
| G5 | Tet | GTGATAATACAGATACCGAAGTAGGGGTTGAGACGCAATCG | 41 | 109 |
| | RF16 | | | |
| H5 | Tet | AAATAAAGTGATGTATACCGAATTCGATTGCGTCTCAACCC | 41 | 110 |
| | RR16 | | | |
| A6 | Tet | AATTCGGTATACATCACTTTATTTAAAACGATGCCCATTTTGT | 43 | 111 |
| | RF17 | | | |
| B6 | Tet | TTGCGCTGAAAAATAAATAATCAACAAAATGGGCATCGTTTT | 42 | 112 |
| | RR17 | | | |
| C6 | Tet | TGATTATTTATTTTTCAGCGCAATTGATAGGCCAAATTCCCG | 42 | 113 |
| | RF18 | | | |
| D6 | Tet | GCACCCACACCGTTGCGGGAATTTGGCCTATCAA | 34 | 114 |
| | RR18 | | | |
| E6 | Tet | CAACGGTGTGGGTGCTATTTACCGAAAATCGTTTTGGA | 38 | 115 |
| | RF19 | | | |
| F6 | Tet | CCAACCATCATGCTATTCCATCCAAAACGATTTTCGGTAAATA | 43 | 116 |
| | RR19 | | | |
| G6 | Tet | TGGAATAGCATGATGGTTGGCTTTTCATTAGCGGGTCT | 38 | 117 |
| | RF20 | | | |
| H6 | Tet | GAATACTGAGTGTAAAAGACCAAGACCCGCTAATGAAAAG | 40 | 118 |
| | RR20 | | | |
| A7 | Tet | TGGTCTTTTACACTCAGTATTCCAAGCCTTTGTGGCAGG | 39 | 119 |
| | RF21 | | | |
| B7 | Tet | CCCATTTAGTGGCTATTCTTCCTGCCACAAAGGCTTG | 37 | 120 |
| | RR21 | | | |
| C7 | Tet | AAGAATAGCCACTAAATGGGGCGAAAAAACGGCAGT | 36 | 121 |
| | RF22 | | | |
| D7 | Tet | CTGCAATAAATTCGAGCAGTACTGCCGTTTTTTCGC | 36 | 122 |
| | RR22 | | | |
| E7 | Tet | ACTGCTCGAATTTATTGCAGATAGTAGTGCATTTGCCTT | 39 | 123 |
| | RF23 | | | |
| F7 | Tet | ACCTTCAGATATAAACGCTAAAAAGGCAAATGCACTACTAT | 41 | 124 |
| | RR23 | | | |
| G7 | Tet | TTTAGCGTTTATATCTGAAGGTTGGTTAGATTTCCCTGTTTTAA | 44 | 125 |
| | RF24 | | | |
| H7 | Tet | CACCACCAGCCAATAAAATTAAAACAGGGAAATCTAAGCA | 40 | 126 |
| | RR24 | | | |
| A8 | Tet | TTTTATTGGCTGGTGGTGGGATCGCTTTACCTGCA | 35 | 127 |
| | RF25 | | | |
| B8 | Tet | ATAGACATCACTCCCTGTAATGCAGGTAAAGCGATCC | 37 | 128 |
| | RR25 | | | |
| C8 | Tet | TTACAGGGAGTGATGTCTATCCAAACAAAGAGTCATGAGC | 40 | 129 |
| | RF26 | | | |
| D8 | Tet | TCCCTGTAAAGCACCTTGCTCATGACTCTTTGTTTGG | 37 | 130 |
| | RR26 | | | |
| E8 | Tet | AAGGTGCTTTACAGGGATTATTGGTGAGCCTTACCA | 36 | 131 |
| | RF27 | | | |
| F8 | Tet | CAATAACACCGGTTGCATTGGTAAGGCTCACCAATAA | 37 | 132 |
| | RR27 | | | |
| G8 | Tet | ATGCAACCGGTGTTATTGGCCCATTACTGTTTACTGT | 37 | 133 |
| | RF28 | | | |
| H8 | Tet | CCAAATTGGTAGTGAATGATTATAAATAACAGTAAACAGTAATGGGC | 47 | 134 |
| | RR28 | | | |
| A9 | Tet | TATTTATAATCATTCACTACCAATTTGGGATGGCTGGATTTGGATTAT | 48 | 135 |
| | RF29 | | | |
| B9 | Tet | ATACAGTAAAACGCTAAACCAATAATCCAAATCCAGCCATC | 41 | 136 |
| | RR29 | | | |
| C9 | Tet | TGGTTTAGCGTTTTACTGTATTATTATCCTGCTATCGATGACC | 43 | 137 |
| | RF30 | | | |
| D9 | Tet | GCTTGAGGGGTTAACATGAAGGTCATCGATAGCAGGATAATA | 42 | 138 |
| | RR30 | | | |
| E9 | Tet | TTCATGTTAACCCCTCAAGCTCAGGGGAGTAAACAGGAG | 39 | 139 |
| | RF31 | | | |
| F9 | Tet | CTAAGCACTTGTCTCCTGTTTACTCCCCTGA | 31 | 140 |
| | RR31 | | | |

The developer orders the set of custom oligonucleotides TetRF1 through Tet RR31, receives the order, and creates the second component containing the custom set of oligonucleotides, a custom set of reagents, and a custom set of reaction conditions. The second component is used to mix the set of custom oligonucleotides to a final concentration of about 1 0-50 μM, create a reaction mixture with a concentration of sodium salt of about 250 to 500 mM and a concentration of DMSO of about 0 to 5%, and subject the reaction mixture to the following custom set of reaction conditions, which includes a thermal cycling program specific for the TetR gene:

| | |
|---|---|
| Denature | 94.0° C. for 1 second |
| Anneal | 57.4° C. for 30 seconds |
| Extend | 72.0° C. for 8 seconds |
| No. Cycles | 1 cycle |
| Denature | 94.0° C. for 1 second |
| Anneal | 56.6° C. for 30 seconds |
| Extend | 72.0° C. for 10 seconds |
| No. Cycles | 4 cycles |
| Denature | 94.0° C. for 1 second |
| Anneal | 55.8° C. for 30 seconds |
| Extend | 72.0° C. for 12 seconds |
| No. Cycles | 4 cycles |
| Denature | 94.0° C. for 1 second |
| Anneal | 55.0° C. for 30 seconds |
| Extend | 72.0° C. for 16 seconds |
| No. Cycles | 4 cycles |
| Denature | 94.0° C. for 1 second |
| Anneal | 54.2° C. for 30 seconds |
| Extend | 72.0° C. for 20 seconds |
| No. Cycles | 4 cycles |
| Denature | 94.0° C. for 1 second |
| Anneal | 53.4° C. for 30 seconds |
| Extend | 72.0° C. for 24 seconds |
| No. Cycles | 4 cycles |

Although the invention has been described with respect to certain methods and applications, it will be appreciated that a variety of changes and modification may be made without departing from the invention as claimed.

All cited documents, including patents, patent applications, and other publications are incorporated herein by reference in their entirety. In addition, the following publications, to the extent they illustrate teachings useful in practicing the present invention, are incorporated herein by reference: US Patent application publication numbers 20050106606, 20040241650, 20030228602, 20030186301, 20030180782, 20030068633, 20020072061; U.S. Pat. Nos.: 6,670,127, 6,521,427, 6,136,568, 5,333,675, 5,038,852; and articles Stemmer; Crameri, Gene, 164 (1995) 49-53, Lance; Burgin, Frontiers in Drug Design & Discovery, January 2005 vol 1, no 1 pp 297-341(45), “Life, reinvented” Wired Magazine, January 2005, 13.01, BioTechniques 30:249-252 (February 2001), Nucleic Acids Research, 2002, vol 30, no. 10 e43, Nucleic Acids Research, 2003, vol 31, no 22 e143 (Xinxin Gao), Nucleic Acids Research, 2004, vol.32, no 12 e98, Nucleic Acids Research, 2004, vol. 32, no 7 e59, Gene 1988 August 15;68(1):101-7, BioTechniques Vol 9 No 3 (1990), Proc. Natl. Acad. Sci USA Vol 88, pp 4084-4088, May 1991, Biochem Biophys Res Commun. Jul. 9, 1998; 248(1):200-3, Nucleic Acids Research, 2004, vol. 32, webserver issue.

We claim:

1. A custom synthesis kit for producing a desired polynucleotide, wherein the kit comprises:
a computer program for use by a developer in designing a desired polynucleotide from a first polynucleotide or a first polypeptide and preselecting a custom set of oligonucleotides that will assemble to create an assembly product for producing the desired polynucleotide, wherein the skill of the developer ranges from a low level to a high level in the art of polynucleotide synthesis; and,
a means for ordering the custom set of oligonucleotides from an outside source;
wherein, the developer is not a provider of oligonucleotides or affiliated with such a provider.

2. The kit of claim 1, wherein the assembly product is a high-fidelity assembly product, such that at least 25% of the assembly product produces the desired polynucleotide.

3. The kit of claim 1, wherein the designing includes entering sequence information from the first polypeptide or the first polynucleotide into the first component to generate information selected from a group consisting of repetitive elements, inverted repeats, GC content, restriction sites, stop codons and multiple frames, CPG motifs, methylation patterns, and combinations thereof, about the desired polynucleotide used in the preselecting of the set of custom oligonucleotides.

4. The kit of claim 1, wherein the computer program further preselects a set of custom reaction conditions that are generated in the form of a digital display, a printout, and/or a computer file or program to be used to instruct a thermocycler to implement the set of custom synthesis reaction conditions.

5. The kit of claim 1, wherein the kit is designed for use by persons having a low level of skill in the art of polynucleotide synthesis.

6. The kit of claim 1, wherein the desired polynucleotide is produced using a single-pot assembly of the assembly product.

7. A system for producing a desired polynucleotide using the custom synthesis kit of claim 1, wherein the system comprises:
a first component comprising the kit; and
a second component designed by the developer to specifically complement the first component, the second component comprising the set of custom oligonucleotides, a set of custom reagents, and a set of custom synthesis reaction conditions for denaturing annealing, and extension, to produce the assembly product using a thermocycler.

8. The system of claim 7, wherein the set of custom oligonucleotides assembles to form a high-fidelity assembly product, such that at least 25% of the assembly product produces the desired polynucleotide.

9. The system of claim 7, wherein the set of custom synthesis reaction conditions are in the form of a digital display, a printout, and/or a computer file or program used to instruct a thermocycler to implement the set of custom synthesis reaction conditions.

10. The system of claim 7, wherein the kit is designed for use by persons having a low level of skill in the art of polynucleotide synthesis.

11. The system of claim 7, wherein the desired polynucleotide is produced using a single-pot assembly of the assembly product.

12. A method of producing a polynucleotide with the custom synthesis kit of claim 1, comprising:
using the kit for the designing of the desired polynucleotide from the first polynucleotide or the first polypeptide and the preselecting of the set of custom oligonucleotides;
wherein, the designing includes entering sequence information from the first polypeptide or the first polynucleotide into the computer program to generate information about the desired polynucleotide;
ordering the custom set of oligonucleotides from an outside source; and,
producing the desired polynucleotide with a thermocycler.

13. The method of claim 12, wherein the designing includes selecting a modification to the first polynucleotide or first polypeptide, and the modification is selected from a group consisting of a point mutation, a variant, a chimeric construction, a codon bias of a host cell, a sequence length, and a combination thereof, such that the desired polynucleotide provides a specific expression system.

14. The method of claim 12, wherein the kit is designed for use by persons having a low level of skill in the art of polynucleotide synthesis.


SEQUENCE LISTING (140 sequences)

SEQ ID NO: 1 - DNA, 432 bp, Unknown Organism
  Description of Unknown Organism DNA sequence
gcattctgaggcattctctaacaggttctcgaccctccgccatggccccgtggatgcatctcctcaccgtgctggccctgctggccctctggggacccaactctgttcaggcctattccagccagcacctgtgcggctccaacctagtggaggcactgtacatgacatgtggacggagtggcttctatagaccccacgaccgccgagagctggaggacctccaggtggagcaggcagaactgggtctggaggcaggcggcctgcagccttcggccctggagatgattctgcagaagcgcggcattgtggatcagtgctgtaataacatttgcacatttaaccagctgcagaactactgcaatgtcccttagacacctgccttgggcctggcctgctgctctgccctggcaaccaataaaccccttgaatgag

SEQ ID NO: 2 - DNA, 25 bp, Unknown Organism
  Description of Unknown Organism DNA sequence
agcattctgaggcattctctaacag

SEQ ID NO: 3 - DNA, 26 bp, Unknown Organism
  Description of Unknown Organism DNA sequence
agagtaagttccccaaataaccaacg

SEQ ID NO: 4 - DNA, 26 bp, Unknown Organism
  Description of Unknown Organism DNA sequence
gcattctgaggcattctctaacaggt

SEQ ID NO: 5 - DNA, 31 bp, Unknown Organism
  Description of Unknown Organism DNA sequence
cgcctcccagctcttggacaatctcttacgg

SEQ ID NO: 6 - DNA, 27 bp, Unknown Organism
  Description of Unknown Organism DNA sequence
tctcgaccctccgccatggccccgtgg

SEQ ID NO: 7 - DNA, 29 bp, Unknown Organism
  Description of Unknown Organism DNA sequence
gccactcctctacgtaggtgccccggtac

SEQ ID NO: 8 - DNA, 30 bp, Unknown Organism
  Description of Unknown Organism DNA sequence
atgcatctcctcaccgtgctggccctgctg

SEQ ID NO: 9 - DNA, 28 bp, Unknown Organism
  Description of Unknown Organism DNA sequence
ccaggggtctcccggtcgtcccggtcgt

SEQ ID NO: 10 - DNA, 30 bp, Unknown Organism
  Description of Unknown Organism DNA sequence
gccctctggggacccaactctgttcaggcc

SEQ ID NO: 11 - DNA, 32 bp, Unknown Organism
  Description of Unknown Organism DNA sequence
ccacgaccgaccttatccggacttgtctcaac

SEQ ID NO: 12 - DNA, 30 bp, Unknown Organism
  Description of Unknown Organism DNA sequence
tattccagccagcacctgtgcggctccaac

SEQ ID NO: 13 - DNA, 30 bp, Unknown Organism
  Description of Unknown Organism DNA sequence
tgtcacggaggtgatccaacctcggcgtgt

SEQ ID NO: 14 - DNA, 33 bp, Unknown Organism
  Description of Unknown Organism DNA sequence
ctagtggaggcactgtacatgacatgtggacgg

SEQ ID NO: 15 - DNA, 35 bp, Unknown Organism
  Description of Unknown Organism DNA sequence
cccagatatcttcggtgaggcaggtgtacagtaca

SEQ ID NO: 16 - DNA, 31 bp, Unknown Organism
  Description of Unknown Organism DNA sequence
agtggcttctatagaccccacgaccgccgag

SEQ ID NO: 17 - DNA, 28 bp, Unknown Organism
  Description of Unknown Organism DNA sequence
cctccaggaggtcgagagccgccagcac

SEQ ID NO: 18 - DNA, 28 bp, Unknown Organism
  Description of Unknown Organism DNA sequence
agctggaggacctccaggtggagcaggc

SEQ ID NO: 19 - DNA, 29 bp, Unknown Organism
  Description of Unknown Organism DNA sequence
gaggtctgggtcaagacggacgaggtgga

SEQ ID NO: 20 - DNA, 28 bp, Unknown Organism
  Description of Unknown Organism DNA sequence
agaactgggtctggaggcaggcggcctg

SEQ ID NO: 21 - DNA, 25 bp, Unknown Organism
  Description of Unknown Organism DNA sequence
cccggcttccgacgtccggcggacg

SEQ ID NO: 22 - DNA, 32 bp, Unknown Organism
  Description of Unknown Organism DNA sequence
cagccttcggccctggagatgattctgcagaa

SEQ ID NO: 23 - DNA, 32 bp, Unknown Organism
  Description of Unknown Organism DNA sequence
gtgttacggcgcgaagacgtcttagtagaggt

SEQ ID NO: 24 - DNA, 33 bp, Unknown Organism
  Description of Unknown Organism DNA sequence
gcgcggcattgtggatcagtgctgtaataacat

SEQ ID NO: 25 - DNA, 38 bp, Unknown Organism
  Description of Unknown Organism DNA sequence
tcgaccaatttacacgtttacaataatgtcgtgactag

SEQ ID NO: 26 - DNA, 35 bp, Unknown Organism
  Description of Unknown Organism DNA sequence
ttgcacatttaaccagctgcagaactactgcaatg

SEQ ID NO: 27 - DNA, 34 bp, Unknown Organism
  Description of Unknown Organism DNA sequence
ccgtccacagattccctgtaacgtcatcaagacg

SEQ ID NO: 28 - DNA, 30 bp, Unknown Organism
  Description of Unknown Organism DNA sequence
tcccttagacacctgccttgggcctggcct

SEQ ID NO: 29 - DNA, 26 bp, Unknown Organism
  Description of Unknown Organism DNA sequence
tcccgtctcgtcgtccggtccgggtt

SEQ ID NO: 30 - DNA, 30 bp, Unknown Organism
  Description of Unknown Organism DNA sequence
gctgctctgccctggcaaccaataaacccc

SEQ ID NO: 31 - DNA, 26 bp, Unknown Organism
  Description of Unknown Organism DNA sequence
gagtaagttccccaaataaccaacgg

SEQ ID NO: 32 - DNA, 717 bp, Unknown Organism
  Description of Unknown Organism DNA sequence
atgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcggttatggtgttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaatag

SEQ ID NO: 33 - DNA, 28 bp, Unknown Organism
  Description of Unknown Organism DNA sequence
aatgagtaaaggagaagaacttttcact

SEQ ID NO: 34 - DNA, 30 bp, Unknown Organism
  Description of Unknown Organism DNA sequence
agataaacatatcaagtaggtacggtacac

SEQ ID NO: 35 - DNA, 20 bp, Unknown Organism
  Description of Unknown Organism DNA sequence
actacaagacacgtgctgaa

SEQ ID NO: 36 - DNA, 25 bp, Unknown Organism
  Description of Unknown Organism DNA sequence
cacaggttcttacaaaggtagaaga

SEQ ID NO: 37 - DNA, 28 bp, Unknown Organism
  Description of Unknown Organism DNA sequence
atgagtaaaggagaagaacttttcactg

SEQ ID NO: 38 - DNA, 37 bp, Unknown Organism
  Description of Unknown Organism DNA sequence
gttcttaaccctgttgaggtcacttttcaagaagagg

SEQ ID NO: 39 - DNA, 41 bp, Unknown Organism
  Description of Unknown Organism DNA sequence
gagttgtcccaattcttgttgaattagatggtgatgttaat

SEQ ID NO: 40 - DNA, 40 bp, Unknown Organism
  Description of Unknown Organism DNA sequence
tgtcttttaaacacgggtaattgtagtggtagattaagtt

SEQ ID NO: 41 - DNA, 32 bp, Unknown Organism
  Description of Unknown Organism DNA sequence
gggcacaaattttctgtcagtggagagggtga

SEQ ID NO: 42 - DNA, 32 bp, Unknown Organism
  Description of Unknown Organism DNA sequence
gcatacaacgtagtggaagtgggagaggtgac

SEQ ID NO: 43 - DNA, 41 bp, Unknown Organism
  Description of Unknown Organism DNA sequence
aggtgatgcaacatacggaaaacttacccttaaatttattt

SEQ ID NO: 44 - DNA, 42 bp, Unknown Organism
  Description of Unknown Organism DNA sequence
catcaaaaggtcatcacgtttatttaaattcccattcaaaag

SEQ ID NO: 45 - DNA, 34 bp, Unknown Organism
  Description of Unknown Organism DNA sequence
gcactactggaaaactacctgttccatggccaac

SEQ ID NO: 46 - DNA, 34 bp, Unknown Organism
  Description of Unknown Organism DNA sequence
ggctttcatcactgttcacaaccggtaccttgtc

SEQ ID NO: 47 - DNA, 38 bp, Unknown Organism
  Description of Unknown Organism DNA sequence
acttgtcactactttcggttatggtgttcaatgctttg

SEQ ID NO: 48 - DNA, 38 bp, Unknown Organism
  Description of Unknown Organism DNA sequence
atactagacccatagagcgtttcgtaacttgtggtatt

SEQ ID NO: 49 - DNA, 36 bp, Unknown Organism
  Description of Unknown Organism DNA sequence
cgagatacccagatcatatgaaacagcatgactttt

SEQ ID NO: 50 - DNA, 34 bp, Unknown Organism
  Description of Unknown Organism DNA sequence
ccgtaccgtgagaactttttcagtacgacaaagt

SEQ ID NO: 51 - DNA, 35 bp, Unknown Organism
  Description of Unknown Organism DNA sequence
tcaagagtgccatgcccgaaggttatgtacaggaa

SEQ ID NO: 52 - DNA, 43 bp, Unknown Organism
  Description of Unknown Organism DNA sequence
cagtagaaactttttatatcaagaaaggacatgtattggaagc

SEQ ID NO: 53 - DNA, 41 bp, Unknown Organism
  Description of Unknown Organism DNA sequence
agaactatatttttcaaagatgacgggaactacaagacacg

SEQ ID NO: 54 - DNA, 37 bp, Unknown Organism
  Description of Unknown Organism DNA sequence
gaagtttgaactgaagtcgtgcacagaacatcaaggg

SEQ ID NO: 55 - DNA, 41 bp, Unknown Organism
  Description of Unknown Organism DNA sequence
tgctgaagtcaagtttgaaggtgatacccttgttaatagaa

SEQ ID NO: 56 - DNA, 41 bp, Unknown Organism
  Description of Unknown Organism DNA sequence
tagttatggaaaattgagctaagataattgttcccatagtg

SEQ ID NO: 57 - DNA, 42 bp, Unknown Organism
  Description of Unknown Organism DNA sequence
tcgagttaaaaggtattgattttaaagaagatggaaacattc

SEQ ID NO: 58 - DNA, 42 bp, Unknown Organism
  Description of Unknown Organism DNA sequence
cataaggttaaacacaggttcttacaaaggtagaagaaattt

SEQ ID NO: 59 - DNA, 40 bp, Unknown Organism
  Description of Unknown Organism DNA sequence
ttggacacaaattggaatacaactataactcacacaatgt

SEQ ID NO: 60 - DNA, 38 bp, Unknown Organism
  Description of Unknown Organism DNA sequence
aacagacggtactacatatgtaacacactcaatatcaa

SEQ ID NO: 61 - DNA, 38 bp, Unknown Organism
  Description of Unknown Organism DNA sequence
atacatcatggcagacaaacaaaagaatggaatcaaag

SEQ ID NO: 62 - DNA, 44 bp, Unknown Organism
  Description of Unknown Organism DNA sequence
acaacacagattaaaacttcaattgaaactaaggtaagaaaaca

SEQ ID NO: 63 - DNA, 40 bp, Unknown Organism
  Description of Unknown Organism DNA sequence
ttaacttcaaaattagacacaacattgaagatggaagcgt

SEQ ID NO: 64 - DNA, 35 bp, Unknown Organism
  Description of Unknown Organism DNA sequence
tattaccagacgatcaacttgcgaaggtagaagtt

SEQ ID NO: 65 - DNA, 39 bp, Unknown Organism
  Description of Unknown Organism DNA sequence
tcaactagcagaccattatcaacaaaatactccaattgg

SEQ ID NO: 66 - DNA, 34 bp, Unknown Organism
  Description of Unknown Organism DNA sequence
cctgtcccggtagcggttaacctcataaaacaac

SEQ ID NO: 67 - DNA, 34 bp, Unknown Organism
  Description of Unknown Organism DNA sequence
cgatggccctgtccttttaccagacaaccattac

SEQ ID NO: 68 - DNA, 37 bp, Unknown Organism
  Description of Unknown Organism DNA sequence
cgtctaacacacctgtccattaccaacagaccatttt

SEQ ID NO: 69 - DNA, 34 bp, Unknown Organism
  Description of Unknown Organism DNA sequence
ctgtccacacaatctgccctttcgaaagatccca

SEQ ID NO: 70 - DNA, 34 bp, Unknown Organism
  Description of Unknown Organism DNA sequence
caccagagagaaaagcaaccctagaaagctttcc

SEQ ID NO: 71 - DNA, 34 bp, Unknown Organism
  Description of Unknown Organism DNA sequence
acgaaaagagagaccacatggtccttcttgagtt

SEQ ID NO: 72 - DNA, 33 bp, Unknown Organism
  Description of Unknown Organism DNA sequence
gggtcgtcgacaatgtttgagttcttcctggta

SEQ ID NO: 73 - DNA, 33 bp, Unknown Organism
  Description of Unknown Organism DNA sequence
tgtaacagctgctgggattacacatggcatgga

SEQ ID NO: 74 - DNA, 33 bp, Unknown Organism
  Description of Unknown Organism DNA sequence
gataaacatatcaagtaggtacggtacacatta

SEQ ID NO: 75 - DNA, 1024 bp, Unknown Organism
  Description of Unknown Organism DNA sequence
atgaatagttcgacaaagatcgcattggtaattacgttactcgatgccatggggattggccttatcatgccagtcttgccaacgttattacgtgaatttattgcttcggaagatatcgctaaccactttggcgtattgcttgcactttatgcgttaatgcaggttatctttgctccttggcttggaaaaatgtctgaccgatttggtcggcgcccagtgctgttgttgtcattaataggcgcatcgctggattacttattgctggctttttcaagtgcgctttggatgctgtatttaggccgtttgctttcagggatcacaggagctactggggctgtcgcggcatcggtcattgccgataccacctcagcttctcaacgcgtgaagtggttcggttggttaggggcaagttttgggcttggtttaatagcggggcctattattggtggttttgcaggagagatttcaccgcatagtcccttttttatcgctgcgttgctaaatattgtcactttccttgtggttatgttttggttccgtgaaaccaaaaatacacgtgataatacagataccgaagtaggggttgagacgcaatcgaattcggtatacatcactttatttaaaacgatgcccattttgttgattatttatttttcagcgcaattgataggccaaattcccgcaacggtgtgggtgctatttaccgaaaatcgttttggatggaatagcatgatggttggcttttcattagcgggtcttggtcttttacactcagtattccaagcctttgtggcaggaagaatagccactaaatggggcgaaaaaacggcagtactgctcgaatttattgcagatagtagtgcatttgcctttttagcgtttatatctgaaggttggttagatttccctgttttaattttattggctggtggtgggatcgctttacctgcattacagggagtgatgtctatccaaacaaagagtcatgagcaaggtgctttacagggattattggtgagcctta

SEQ ID NO: 76 - DNA, 25 bp, Unknown Organism
  Description of Unknown Organism DNA sequence
aatgaatagttcgacaaagatcgca

SEQ ID NO: 77 - DNA, 25 bp, Unknown Organism
  Description of Unknown Organism DNA sequence
actaagcacttgtctcctgtttact

SEQ ID NO: 78 - DNA, 25 bp, Unknown Organism
  Description of Unknown Organism DNA sequence
acacgtgataatacagataccgaag

SEQ ID NO: 79 - DNA, 35 bp, Unknown Organism
  Description of Unknown Organism DNA sequence
atgaatagttcgacaaagatcgcattggtaattac

SEQ ID NO: 80 - DNA, 40 bp, Unknown Organism
  Description of Unknown Organism DNA sequence
cccatggcatcgagtaacgtaattaccaatgcgatctttg

SEQ ID NO: 81 - DNA, 36 bp, Unknown Organism
  Description of Unknown Organism DNA sequence
gttactcgatgccatggggattggccttatcatgcc

SEQ ID NO: 82 - DNA, 38 bp, Unknown Organism
  Description of Unknown Organism DNA sequence
gtaataacgttggcaagactggcatgataaggccaatc

SEQ ID NO: 83 - DNA, 41 bp, Unknown Organism
  Description of Unknown Organism DNA sequence
agtcttgccaacgttattacgtgaatttattgcttcggaag

SEQ ID NO: 84 - DNA, 40 bp, Unknown Organism
  Description of Unknown Organism DNA sequence
ccaaagtggttagcgatatcttccgaagcaataaattcac

SEQ ID NO: 85 - DNA, 39 bp, Unknown Organism
  Description of Unknown Organism DNA sequence
atatcgctaaccactttggcgtattgcttgcactttatg

SEQ ID NO: 86 - DNA, 41 bp, Unknown Organism
  Description of Unknown Organism DNA sequence
caaagataacctgcattaacgcataaagtgcaagcaatacg

SEQ ID NO: 87 - DNA, 39 bp, Unknown Organism
  Description of Unknown Organism DNA sequence
cgttaatgcaggttatctttgctccttggcttggaaaaa

SEQ ID NO: 88 - DNA, 35 bp, Unknown Organism
  Description of Unknown Organism DNA sequence
accaaatcggtcagacatttttccaagccaaggag

SEQ ID NO: 89 - DNA, 29 bp, Unknown Organism
  Description of Unknown Organism DNA sequence
tgtctgaccgatttggtcggcgcccagtg

SEQ ID NO: 90 - DNA, 34 bp, Unknown Organism
  Description of Unknown Organism DNA sequence
cgcctattaatgacaacaacagcactgggcgccg

SEQ ID NO: 91 - DNA, 43 bp, Unknown Organism
  Description of Unknown Organism DNA sequence
ctgttgttgtcattaataggcgcatcgctggattacttattgc

SEQ ID NO: 92 - DNA, 39 bp, Unknown Organism
  Description of Unknown Organism DNA sequence
gcgcacttgaaaaagccagcaataagtaatccagcgatg

SEQ ID NO: 93 - DNA, 39 bp, Unknown Organism
  Description of Unknown Organism DNA sequence
tggctttttcaagtgcgctttggatgctgtatttaggcc

SEQ ID NO: 94 - DNA, 40 bp, Unknown Organism
  Description of Unknown Organism DNA sequence
gtgatccctgaaagcaaacggcctaaatacagcatccaaa

SEQ ID NO: 95 - DNA, 34 bp, Unknown Organism
  Description of Unknown Organism DNA sequence
gtttgctttcagggatcacaggagctactggggc

SEQ ID NO: 96 - DNA, 29 bp, Unknown Organism
  Description of Unknown Organism DNA sequence
ccgatgccgcgacagccccagtagctcct

SEQ ID NO: 97 - DNA, 32 bp, Unknown Organism
  Description of Unknown Organism DNA sequence
tgtcgcggcatcggtcattgccgataccacct

SEQ ID NO: 98 - DNA, 33 bp, Unknown Organism
  Description of Unknown Organism DNA sequence
cgcgttgagaagctgaggtggtatcggcaatga

SEQ ID NO: 99 - DNA, 33 bp, Unknown Organism
  Description of Unknown Organism DNA sequence
cagcttctcaacgcgtgaagtggttcggttggt

SEQ ID NO: 100 - DNA, 35 bp, Unknown Organism
  Description of Unknown Organism DNA sequence
cccaaaacttgcccctaaccaaccgaaccacttca

SEQ ID NO: 101 - DNA, 35 bp, Unknown Organism
  Description of Unknown Organism DNA sequence
taggggcaagttttgggcttggtttaatagcgggg

SEQ ID NO: 102 - DNA, 39 bp, Unknown Organism
  Description of Unknown Organism DNA sequence
tgcaaaaccaccaataataggccccgctattaaaccaag

SEQ ID NO: 103 - DNA, 38 bp, Unknown Organism
  Description of Unknown Organism DNA sequence
cctattattggtggttttgcaggagagatttcaccgca

SEQ ID NO: 104 - DNA, 37 bp, Unknown Organism
  Description of Unknown Organism DNA sequence
cagcgataaaaaagggactatgcggtgaaatctctcc

SEQ ID NO: 105 - DNA, 41 bp, Unknown Organism
  Description of Unknown Organism DNA sequence
tagtcccttttttatcgctgcgttgctaaatattgtcactt

SEQ ID NO: 106 - DNA, 41 bp, Unknown Organism
  Description of Unknown Organism DNA sequence
ccaaaacataaccacaaggaaagtgacaatatttagcaacg

SEQ ID NO: 107 - DNA, 42 bp, Unknown Organism
  Description of Unknown Organism DNA sequence
tccttgtggttatgttttggttccgtgaaaccaaaaatacac

SEQ ID NO: 108 - DNA, 46 bp, Unknown Organism
  Description of Unknown Organism DNA sequence
ctacttcggtatctgtattatcacgtgtatttttggtttcacggaa

SEQ ID NO: 109 - DNA, 41 bp, Unknown Organism
  Description of Unknown Organism DNA sequence
gtgataatacagataccgaagtaggggttgagacgcaatcg

SEQ ID NO: 110 - DNA, 41 bp, Unknown Organism
  Description of Unknown Organism DNA sequence
aaataaagtgatgtataccgaattcgattgcgtctcaaccc

SEQ ID NO: 111 - DNA, 43 bp, Unknown Organism
  Description of Unknown Organism DNA sequence
aattcggtatacatcactttatttaaaacgatgcccattttgt

SEQ ID NO: 112 - DNA, 42 bp, Unknown Organism
  Description of Unknown Organism DNA sequence
ttgcgctgaaaaataaataatcaacaaaatgggcatcgtttt

SEQ ID NO: 113 - DNA, 42 bp, Unknown Organism
  Description of Unknown Organism DNA sequence
tgattatttatttttcagcgcaattgataggccaaattcccg

SEQ ID NO: 114 - DNA, 34 bp, Unknown Organism
  Description of Unknown Organism DNA sequence
gcacccacaccgttgcgggaatttggcctatcaa

SEQ ID NO: 115 - DNA, 38 bp, Unknown Organism
  Description of Unknown Organism DNA sequence
caacggtgtgggtgctatttaccgaaaatcgttttgga

SEQ ID NO: 116 - DNA, 43 bp, Unknown Organism
  Description of Unknown Organism DNA sequence
ccaaccatcatgctattccatccaaaacgattttcggtaaata

SEQ ID NO: 117 - DNA, 38 bp, Unknown Organism
  Description of Unknown Organism DNA sequence
tggaatagcatgatggttggcttttcattagcgggtct

SEQ ID NO: 118 - DNA, 40 bp, Unknown Organism
  Description of Unknown Organism DNA sequence
gaatactgagtgtaaaagaccaagacccgctaatgaaaag

SEQ ID NO: 119 - DNA, 39 bp, Unknown Organism
  Description of Unknown Organism DNA sequence
tggtcttttacactcagtattccaagcctttgtggcagg

SEQ ID NO: 120 - DNA, 37 bp, Unknown Organism
  Description of Unknown Organism DNA sequence
cccatttagtggctattcttcctgccacaaaggcttg

SEQ ID NO: 121 - DNA, 36 bp, Unknown Organism
  Description of Unknown Organism DNA sequence
aagaatagccactaaatggggcgaaaaaacggcagt

SEQ ID NO: 122 - DNA, 36 bp, Unknown Organism
  Description of Unknown Organism DNA sequence
ctgcaataaattcgagcagtactgccgttttttcgc

SEQ ID NO: 123 - DNA, 39 bp, Unknown Organism
  Description of Unknown Organism DNA sequence
actgctcgaatttattgcagatagtagtgcatttgcctt

SEQ ID NO: 124 - DNA, 41 bp, Unknown Organism
  Description of Unknown Organism DNA sequence
accttcagatataaacgctaaaaaggcaaatgcactactat

SEQ ID NO: 125 - DNA, 44 bp, Unknown Organism
  Description of Unknown Organism DNA sequence
tttagcgtttatatctgaaggttggttagatttccctgttttaa

SEQ ID NO: 126 - DNA, 40 bp, Unknown Organism
  Description of Unknown Organism DNA sequence
caccaccagccaataaaattaaaacagggaaatctaacca

SEQ ID NO: 127 - DNA, 35 bp, Unknown Organism
  Description of Unknown Organism DNA sequence
ttttattggctggtggtgggatcgctttacctgca

SEQ ID NO: 128 - DNA, 37 bp, Unknown Organism
  Description of Unknown Organism DNA sequence
atagacatcactccctgtaatgcaggtaaagcgatcc

SEQ ID NO: 129 - DNA, 40 bp, Unknown Organism
  Description of Unknown Organism DNA sequence
ttacagggagtgatgtctatccaaacaaagagtcatgagc

SEQ ID NO: 130 - DNA, 37 bp, Unknown Organism
  Description of Unknown Organism DNA sequence
tccctgtaaagcaccttgctcatgactctttgtttgg

SEQ ID NO: 131 - DNA, 36 bp, Unknown Organism
  Description of Unknown Organism DNA sequence
aaggtgctttacagggattattggtgagccttacca

SEQ ID NO: 132 - DNA, 37 bp, Unknown Organism
  Description of Unknown Organism DNA sequence
caataacaccggttgcattggtaaggctcaccaataa

SEQ ID NO: 133 - DNA, 37 bp, Unknown Organism
  Description of Unknown Organism DNA sequence
atgcaaccggtgttattggcccattactgtttactgt

SEQ ID NO: 134 - DNA, 47 bp, Unknown Organism
  Description of Unknown Organism DNA sequence
ccaaattggtagtgaatgattataaataacagtaaacagtaatgggc

SEQ ID NO: 135 - DNA, 48 bp, Unknown Organism
  Description of Unknown Organism DNA sequence
tatttataatcattcactaccaatttgggatggctggatttggattat

SEQ ID NO: 136 - DNA, 41 bp, Unknown Organism
  Description of Unknown Organism DNA sequence
atacagtaaaacgctaaaccaataatccaaatccagccatc

SEQ ID NO: 137 - DNA, 43 bp, Unknown Organism
  Description of Unknown Organism DNA sequence
tggtttagcgttttactgtattattatcctgctatcgatgacc

SEQ ID NO: 138 - DNA, 42 bp, Unknown Organism
  Description of Unknown Organism DNA sequence
gcttgaggggttaacatgaaggtcatcgatagcaggataata

SEQ ID NO: 139 - DNA, 39 bp, Unknown Organism
  Description of Unknown Organism DNA sequence
ttcatgttaacccctcaagctcaggggagtaaacaggag

SEQ ID NO: 140 - DNA, 31 bp, Unknown Organism
  Description of Unknown Organism DNA sequence
ctaagcacttgtctcctgtttactcccctga